(19) United States
(12) Patent Application Publication
(10) Pub. No.: US 2014/0163495 A1
(43) Pub. Date: Jun. 12, 2014

(54) Washable stoma wafer and elastic barrier ring recycling application method

(71) Applicant: Achilles Nassopoulos, Montreal (CA)

(72) Inventor: Achilles Nassopoulos, Montreal (CA)

(21) Appl. No.: 13/707,870

(22) Filed: Dec. 7, 2012

(51) Int. Cl. A61F 5/443

(52) U.S. Cl. CPC A61F 5/443
USPC 604/338

(57) ABSTRACT

A method of use of colostomy, ileostomy, urostomy or similar ostomy stoma wafers produced as a final product of use without adhesive backing equipped either with plastic type wafer centers with an innovative center elastic stoma barrier ring or with completely innovative one piece elastic stoma wafer centers both of which plastic or elastic centers are surrounded by washable flexible material, to which adhesive is applied, are applied on a stoma, are removed after use, are washed with hand soap or other cleaning agent, are dried, are coated with fresh adhesive are applied on a stoma again and to repeat the process until the stoma wafers become unusable, or the stoma wafers are washed while adhering on the skin around a stoma of a patient and adhesive is re-applied wherever lost and to repeat the process until the stoma wafers become unusable.

### CROSS-REFERENCE TO RELATED APPLICATIONS

| US. Patent No. | Date | Applicant |
|---|---|---|
| 4,231,369 | Nov. 4, 1980 | Sarensen et al. |
| 4,253,460 | Mar. 3, 1981 | Chen et al |
| 4,324,595 | Apr. 13, 1982 | Kasprzak |
| 4,642,107 | Feb. 10, 1987 | Arnone et al. |
| 4,681,574 | Jul. 21, 1987 | Eastman |
| 4,846,798 | Jul. 11, 1989 | Holtermann et al. |
| 5,051,259 | Sep. 24, 1991 | Olsen et al. |
| 5,520,670 | May 28, 1996 | Blum |
| 6,206,864 B1 | Mar. 27, 2001 | Kavanagh et al |
| 6,520,943 B1 | Feb. 18, 2001 | Wagner |
| 6,764,473 B2 | Jul. 20, 2004 | Nielsen et al |
| US Pub. No. | Jun. 10, 2004 | McKissick |
| US 2004/0111072 A1 | | |
| 683663 B1 | Mar. 8, 2005 | Mills et al |
| 7,586,019 B2 | Sep. 8, 2009 | Oclund et al |

### BACKGROUND OF THE INVENTION

Colostomy is the surgical procedure of making an opening from the colon to the outside of the abdomen. In a colostomy, the surgeon may remove a segment of the colon, which results in the colon to be in two separate parts. One end of the colon is passed through a small hole in the abdominal wall. This section of intestine called a stoma, allows for body waste to leave the body from the colon. The other end of the colon, which is attached to the rectum, may either be removed or closed off with sutures and left in the abdomen. There are other similar stoma operations such as ileostomy which is a stoma from the small intestine, and urostomy which is a stoma from the urinary system. Operations are performed for temporary or permanent stomas.

Such operations are performed on humans as well as animals. In all cases, stoma wafers are used which are adhesive skin barriers or skin flanges with an opening at the center to allow the stoma to protrude. There are wafers that require cutting the center opening to custom fit the size of the stoma, there are wafers with a small opening of stretchable material at the center to custom fit to the stoma, and there are many more types of wafers for varied applications and conditions of stomas. One of the main goals of most stoma wafers is to prevent the contact of body waste with the skin. These stoma wafers are either a one piece wafer and bag or a two piece wafer with a snap-on or similar attachment for the bags. The bag serves to hold the body waste. Many other products related to stomas in preventing body waste leaks on the skin, or in protecting the skin from leaks, or in cleaning the adhesive from the skin or in treating skin irritations due to the contact of body waste with the skin are in use and many more items are being produced specifically for stomas.

Conventional colostomy or other types of stoma wafers are supplied with an adhesive or glue backing for ready to use application as in U.S. Pat. No. 4,642,107, No. 4,681,574, No. 5,051,259, No. 6,206,864 B1, and No. 6,764,473 A great number of colostomy patients or patients with similar operations such as ileostomies that eliminate body wastes generally in the liquid form or urostomies that eliminate urine are unable to get a tight fitting wafer and experience leaks, thus forcing them to change the wafer prematurely. If leaks are left unattended skin rashes and irritations will develop which can have severe consequences if they are not treated. The treatment may require the need to leave the stoma to air for prolonged periods of time with special skin irritation reducing products or to seek medical attention.

A brief summary of the products required and the steps for stoma care, specifically for colostomy is presented in a general precise step by step method in the US Pub. No. US 2004/0111072 in the abstract which reads “A display arrangement for organizing and presenting supplies needed for ostomy care has at least one display surface having alphanumerically sequenced indicia disposed thereon. The indicia include at least one indicator for at least one skin preparation product, at least one indicator for stoma measurement, at least one indicator for at least one wafer cutting supplement, at least one indicator for a wafer, at least one indicator for at least one adhesive product which is in the form of paste; and at least one indicator for a pouch.”

There are additional indicia in the detailed explanations and detailed drawings of the Pub. No. US 2004/0111072 that are not included in the abstract, mainly of importance for the purpose of this present patent are the indicator No. 1 which is a trash bag or bin and part of indicator No. 4 which is the adhesive prep. The adhesive prep is presented as an adhesive wipe only however it is also available in an adhesive spray form in the detailed explanations of the same publication. Once the wafer or wafer and bag are removed, they are discarded in the un-recyclable trash bag shown in indicator No. 1 as the wafer or wafer and bag become contaminated with human body wastes.

Publication: No. US 2004/0111072 A1 indicates the following method for ostomy care numbered in the order of the display:

### Indicator Description

1. Trash bag: Have a trash bag or biohazard bag or bin available shown in the display arrangement. (Assuming it is for the used wafer and the towelettes.)
2. Adhesive remover: Use Adhesive Remover Wipe to aid in removing residual adhesive, leaving an oily residue.
3. Towelettes: Use the towelettes as a moist wipe to remove the oily residue on the skin left by the adhesive remover.
4. Adhesive Prep: Apply adhesive prep as adhesive prep wipe or adhesive prep spray on the skin.
5. Pencil: Pencil to trace the circumference of the stoma in the center of the wafer using the Stoma measurement Guide Indicator 6.
6. Measurement guide: Measure the stoma size with the stoma measuring guide and trace with the pencil the size of the stoma measured in the center of the stoma wafer indicator 7.
7. Wafer: Stoma wafer
8. Scissors: Cut in the stoma wafer the circumference traced by the pencil Indicator 5 with the scissors Indicator 8.
9. Paste: Apply the Adhesive Paste to stick the wafer to the patient's skin.
10. Pouch or bag: Apply the pouch to the wafer by joining together.
11. Clip, cap, or plug: Apply the Clip, Cap, or Plug to close the bottom of the bag.

Whereas the present patent indicates the following recycling method for ostomy care in steps:

Method A) Stoma washable wafers with plastic or similar material centers with stoma size diameter cut-out in the center surrounded by flexible material or fabric. onto which adhesive is generally applied.
1. Measure the stoma size with the stoma measuring guide and trace with the pencil the size of the stoma measured in the center plastic inner diameter of the washable stoma wafer.
2. Cut out the center inner plastic diameter of the washable stoma wafer traced with the pencil to the size of the stoma.
3. Apply the skin adhesive glue in the form of liquid or spray that is washable with hand soap, alcohol, stoma adhesive remover, or other cleaning agent to the back of the washable stoma wafer with the center cut out prepared in step 2 above making sure to cover the entire surface including the plastic and the material surrounding the plastic surface with the adhesive to form an adhesive backing.
4. Apply the adhesive prep in the form of liquid or spray on the skin around the stoma.
5. Apply the stoma wafer prepared in step 3 above with the applied adhesive backing that is washable with hand soap, alcohol, stoma adhesive remover, or other cleaning agent, to the prepared skin around the stoma.
6. Measure the stoma size and cut out the center inner diameter of the elastic, or other material with similar elasticity, flat stoma barrier ring to the diameter size of the stoma for a snug fit.
7. Apply the skin adhesive glue or adhesive prep in the form of liquid or spray that is washable with hand soap, alcohol, stoma adhesive remover, or other cleaning agent to the back of the elastic stoma barrier ring prepared in step 6 above, to form an adhesive backing.
8. Apply the adhesive prep in the form of liquid or spray to the skin surface exposed around the stoma center plastic cut-out of the washable stoma wafer now adhering on the skin and apply the same adhesive prep to overlap onto the plastic surface around the washable stoma wafer center cut-out by a surface area at least equal to the width of the elastic stoma barrier ring prepared in step 7 above.
9. Apply the elastic stoma barrier ring prepared in step 7 above around the stoma and adhere to the skin around the stoma and to the washable stoma wafer plastic surface overlapped with adhesive prep in step 8 above.
10. Remove the applied washable stoma wafer and elastic barrier ring after use by physical means and with a stoma adhesive remover and do not discard the used washable stoma wafer.
11. Wash the washable stoma wafer and the elastic stoma barrier ring removed in step 10 above with hand soap, alcohol, stoma adhesive remover or other cleaning agent.
12. Dry the washable stoma wafer and elastic stoma barrier ring.
13. Recycle the washable stoma wafer and the elastic stoma barrier ring dried in step 12 above by starting from step 3 above again where the washable adhesive is applied to the washable stoma wafer down to this present step 13, with the exception of step 5 above since the same elastic stoma barrier ring is used again and to repeat this thirteen (13) step method until the washable stoma wafer and elastic stoma barrier ring become unusable.
14. The washable stoma wafer may be supplied with the stoma pouch or bag as a one-piece wafer and bag assembly or a two-piece wafer and bag supplied as two separate items that connect together by means of a snap-on or other interlocking method or mechanism. The one-piece wafer and bag or bag and wafer supplied separately may either be washed and used again in the same manner as the washable wafer is washed and used again explained in the above method, once the body waste in the stoma bag has been removed and discarded in the W.C. or the same stoma bags may not require washing if “throw away” bag liners that can be flushed in the W.C. such as the throw away liner in U.S. Pat. No. 5,423,782 “Disposable ostomy bag liner” are used inside the stoma bags or pouches.

Note:
a) A trash bag or biohazard bag or bin is not required for the washable stoma wafer and for the elastic stoma barrier ring as they are recyclable. The body waste stoma bag is emptied in the W.C. and washed or the body waste collects in a throw away bag liner that is removed and flushed in the W.C; stoma paste is not used either and therefore does not require a trash bag, however there will still be the adhesive wipes and towelettes that will require a trash bag unless washable sponges are used to apply adhesive remover or adhesive prep solutions supplied in bottle or spray formats.
   and

Method B) Stoma washable wafers with elastic or similar material centers with stoma size diameter cut-out in the center surrounded by flexible material or fabric onto which adhesive is generally applied.

Note: Method B) is identical to Method A) However since the center of the washable elastic stoma wafer is elastic, the elastic stoma barrier ring is not required, thus steps 6) through 9) of method A) are not required and are therefore omitted making the “Method B) a ten (10) step method.

### BRIEF SUMMARY OF THE INVENTION

It is an object of the present invention to reduce stoma body waste leaks and to be more environmentally friendly as well. By improving the effectiveness of existing stoma wafers against leaks through the use of washable recyclable stoma wafers equipped with a plastic (or similar material) or elastic (or similar material) inner center cut-out which is surrounded by flexible material or fabric onto which adhesive is generally applied In the case of the present invention however, the said stoma wafers will be supplied without adhesive or glue on the back as adhesive backing and will also be equipped with stoma barrier rings with elastic properties without adhesive or glue on the back as adhesive backing, for stomas of colostomy, ileostomy, and urostomy patients. The present invention vastly improves the comfort and effectiveness of existing stoma wafers by offering a method of use that:
1. Will be more environmentally friendly and more ecological than existing conventional adhesive-backed stoma wafers that are discarded after their first application or use because their adhesive backing is not washable. The present invention's washable stoma wafer improves on the said adhesive-backed stoma wafers as it is recyclable and reusable by washing the washable stoma wafer after each use to clean off the used adhesive backing and body wastes, and by the application of fresh washable glue or adhesive as adhesive backing to the cleaned washable stoma wafer, thus forming a fresh new adhesive backing to apply on a stoma, and by repeating this recycling method until the washable stoma wafer becomes unusable.
2. Will be more environmentally friendly and more ecological than existing conventional stoma barrier rings that are not washable and are discarded after their first use or application by being recyclable by washing the elastic stoma barrier ring after use to wash off the used adhesive backing and body wastes and by applying fresh new washable glue or adhesive as adhesive backing to the cleaned elastic stoma barrier ring to form a new adhesive backing, to apply on the stoma as a fresh elastic stoma barrier ring on a stoma and to repeat this recycling method until the elastic stoma barrier ring becomes unusable.
3. Will be more environmentally friendly and more ecological than existing conventional stoma wafers by reducing the volume of adhesive backing applied onto stoma wafers by using liquid or spray adhesive only while still being efficient for longer periods of time.
4. Will reduce skin irritation by being more efficient against body waste leaks by using adhesive preps as liquids or adhesive sprays as adhesive backing of the washable wafer thus making it thinner, better fitting and adhering better in cracks and crevices of the skin surface, molding like a second skin more efficiently than conventional stoma wafers that use adhesive paste or other thicker or more dense backings instead that result in unavoidable pleats or lifting of areas of the wafer due to the thickness of the adhesive backing, because uncontrolled pleats or the lifting of areas under the wafer may result in body waste leaks and skin irritation.
5. Will reduce skin irritation by being more efficient against body waste leaks around the center cut-out elastic stoma barrier rings than barrier rings of existing stoma wafers that use pastes instead of barrier rings to seal the exposed skin around the stoma of a cut to stoma size diameter opening at the center cut-out of conventional stoma wafers or use conventional stoma barrier rings to seal the exposed skin around the same stoma of a cut to stoma size diameter opening at the center cut-out of conventional stoma wafers because these conventional barrier rings are made of unwashable, malleable, stretchable material that cannot be brought back to its original shape, are pre-coated with adhesives that generally degrade gradually in the presence of body waste, by using an elastic recyclable stoma barrier ring that will fit snugly around a stoma without damaging the stoma because of the elasticity of the elastic stoma barrier ring, that will remain impermeable to body waste as long as the selected adhesive is still holding, and that will retain its original shape after use for more than one application after washing.
6. Will reduce skin irritation caused by traumatizing the skin around the stoma from the removal of a used stoma wafer, from the cleaning of the stoma area after the removal of the used stoma wafer and from the more frequent replacement of the used stoma wafer, by prolonging the changing of stoma wafers on stomas by preventing body waste leaks from occurring for longer periods of time by adhering in more cracks and crevices and molding better on the skin like a second skin without any pleats resulting in improved adherence for longer periods of time, than conventional stoma wafers with thicker adhesive backings that do not adhere on all surfaces as well and develop leaks sooner, thus requiring to be removed and replaced more frequently.
7. 7. Will reduce skin irritation by the ability to be selective in the glue or adhesive that is used for the adhesive backing of the washable stoma wafer and of the elastic stoma barrier ring that are not already supplied with adhesive or glue, because several soluble adhesive preps or adhesive sprays are efficient in use as adhesive backing and may be easily cleaned with several types of adhesive remover wipes made specifically for stoma applications.
8. Will reduce skin irritation by acting like a second skin in order to better seal the area around the stoma and avoid leaks, thus eliminating the need to add stoma paste to seal open skin cracks, crevices and pleats caused by the thicker conventional adhesive backing that does not bend as well; pastes also tend to be difficult to remove when cleaning without tearing or irritating the skin if the paste has adhered firmly to the skin, even when attempting to remove the paste with the help of adhesive remover solvents or wipes.
9. Will reduce skin irritation by eliminating the need to add stoma paste to seal the exposed skin around the stoma center cut-out of the washable stoma wafer adhering on the skin because the exposed skin will instead be covered by the impermeable washable elastic stoma barrier ring which will remain effectively in place for as long as the adhesive can hold, or if the elastic stoma barrier ring is incorporated with the washable stoma wafer as a permanent attachment, or if the center of the washable stoma wafer is made from elastic or materials with similar elasticity, it will hold for as long as the selected adhesive backing holds the washable stoma wafer with the permanently attached elastic stoma barrier ring or as long as the washable stoma wafer with the elastic center adhere to the skin around the stoma.
10. Will reduce skin irritation by allowing the skin to breathe better through the section of the washable stoma wafer membrane which is a band of washable flexible material or fabric onto which adhesive is generally applied from one (1) to six (6) inches in band width that surrounds the inner plastic or elastic cut-out section, because liquids or sprays will be used as adhesive backings on this surface as opposed to present conventional stoma wafers that use coatings of impermeable adhesive that prevent proper air flow and are not efficient in eliminating heat, causing the skin to sweat or to become humid and the stoma wafer to lift.
11. ill eliminate the need of shaving the hair around the stoma, as this method of applying adhesive on a stoma wafer that does not have an adhesive backing, allows the choice of using a less aggressive adhesive such as one of several stoma adhesive wipe types or stoma adhesive spray types that are effective but that will not break or uproot the hair follicles beneath it. Since the outer membrane of the washable stoma wafer is porous, it allows the adhesive remover to penetrate through and dissolve the adhesive, enabling the washable stoma wafer to simply be peeled off, resulting in a much gentler and less problematic removal of the washable stoma wafer since broken or uprooted hair follicles and shaving often lead to ingrown hair, irritations, infections, or other problems.
12. Will be more sanitary than existing conventional stoma wafers and stoma barrier rings that are not washable and are discarded after their initial use as hazardous unrecyclable contaminated material with body waste possibly adhering to the wafer and to the stoma barrier ring because the body waste on the washable stoma wafer and the elastic stoma barrier ring is washed off and thus neutralized after each use in order to use the washable stoma wafer and elastic stoma barrier ring again with fresh adhesive.
13. Will allow individuals with a stoma to have the privilege of changing the washable stoma wafer and elastic skin barrier ring daily or more often if a reason demands it without the associated cost or the environmentally polluting ecological impact of discarding wafers and stoma barrier rings after their initial use.
14. Will allow individuals with a stoma to have the privilege of bathing or swimming either with the washable stoma wafer still glued on with the stoma pouch or bag attached, or even of exposing the stoma to the elements with just the washable stoma skin wafer glued on as a second skin, without the need of having the stoma pouch or bag attached. This could be accomplished without the risk of the washable stoma wafer becoming unglued because of the fact that the appropriate washable stoma wafer glue or adhesive selected that is water-resistant, can be used without resulting in eventual skin irritations.
15. Will allow individuals with a stoma to have the privilege of bathing to wash the stoma and the skin surface underneath the washable stoma wafer even with mild soap if well rinsed afterwards because the outer membrane of the washable stoma wafer is porous and the skin under the washable stoma wafer is reached without affecting the adhesive nor its adherence, without irritating the skin and without causing other secondary skin effects.

### BRIEF DESCRIPTION OF THE SEVERAL VIEWS OF THE DRAWING

FIG. 1 is a rear view of a one-piece washable colostomy stoma wafer and body waste receiving bag assembly with a “Velcro” type and a clamp type bag emptying and sealing mechanism of the draining pouch specifying the surface area to be covered by the adhesive applied to form an adhesive backing.

FIG. 2 is a front view of a one-piece washable colostomy stoma wafer and body waste receiving bag assembly with a “Velcro” type and a clamp type bag emptying and sealing mechanism of the draining pouch.

FIG. 3 is a rear view of the body waste collecting bag of a two-piece washable colostomy stoma wafer and separate body waste collecting bag assembly with a “Velcro” type and a clamp type bag emptying and sealing mechanism connecting the washable stoma wafer to the body waste collecting bag by the snap-on method as an example of one of several methods of holding together the washable stoma wafer to the separate body waste collecting bag.

FIG. 4 is a front view of the body waste collecting bag of a two-piece washable colostomy stoma wafer and separate body waste collecting bag assembly with a “Velcro” type and a clamp type emptying and sealing mechanism connecting the washable stoma wafer to the body waste collecting bag by the snap on method as an example of one of several methods of holding together the washable stoma wafer to the separate body waste collecting bag.

FIG. 5 is a rear view of the washable colostomy stoma wafer of a two-piece washable colostomy stoma wafer and separate body waste collecting bag assembly with a “Velcro” type and with a clamp type bag emptying and sealing mechanism of the body waste collecting bag, connecting the washable stoma wafer to the body waste collecting bag by the snap-on method as an example of one of several methods of holding together the washable stoma wafer to the separate body waste collecting bag.

FIG. 6 is a front view of the washable colostomy stoma wafer of a two-piece washable colostomy stoma wafer and separate body waste collecting bag assembly with a “Velcro” type bag and with a clamp type bag emptying and sealing mechanism of the body waste collecting bag connecting the washable stoma wafer to the body waste collecting bag by the snap-on method as an example of one of several methods of holding together the washable stoma wafer to the separate body waste collecting bag.

FIG. 7 is a rear view of the elastic stoma barrier ring.

FIG. 8 is a front view of the elastic stoma barrier ring.

FIG. 9 is a nineteen (19) step sketch of the complete method of use of the present patent.

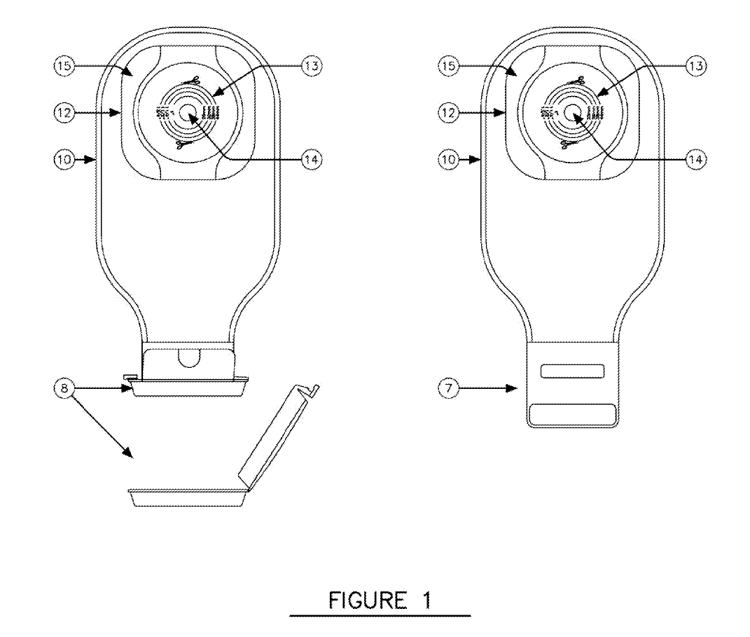

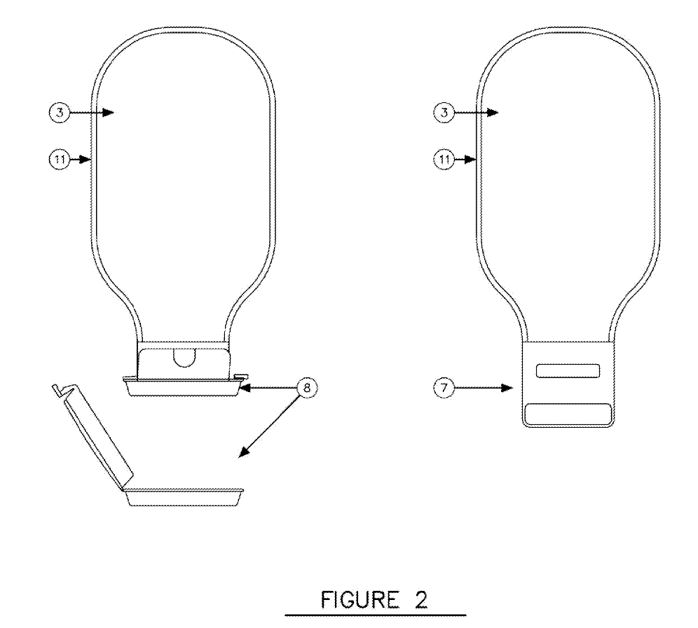

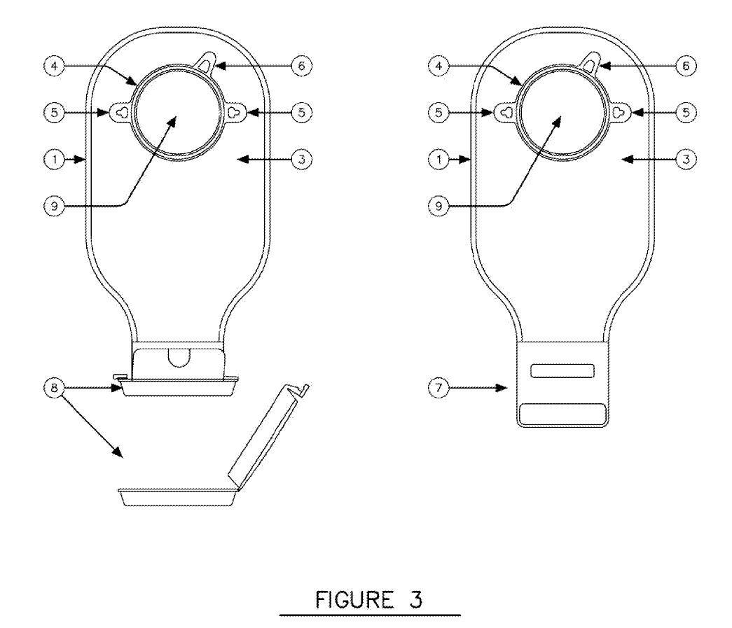

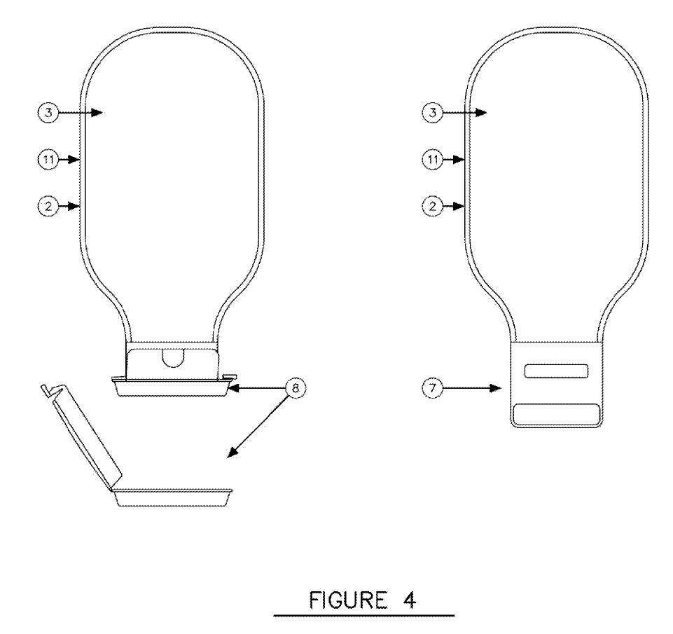

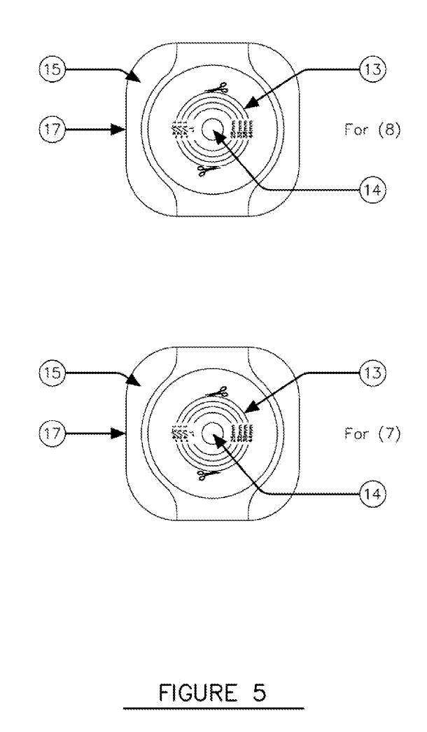

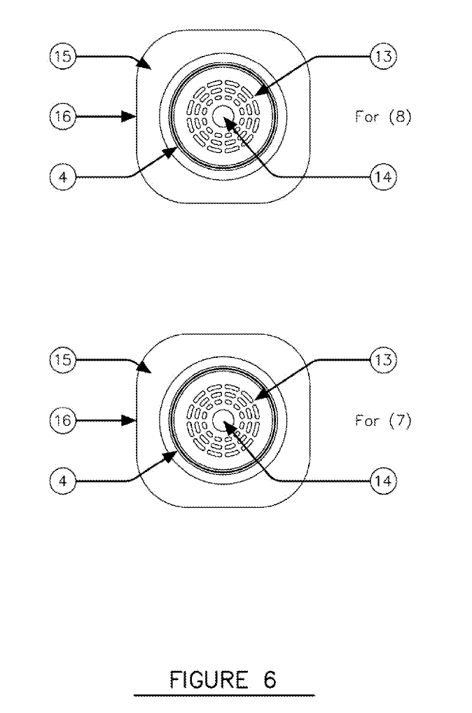

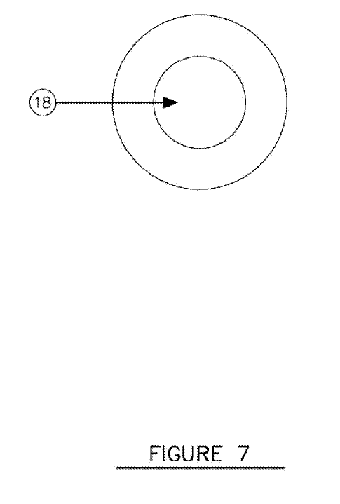

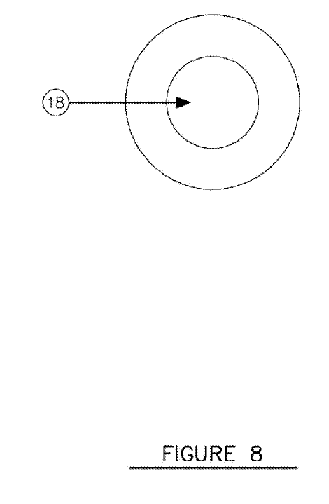

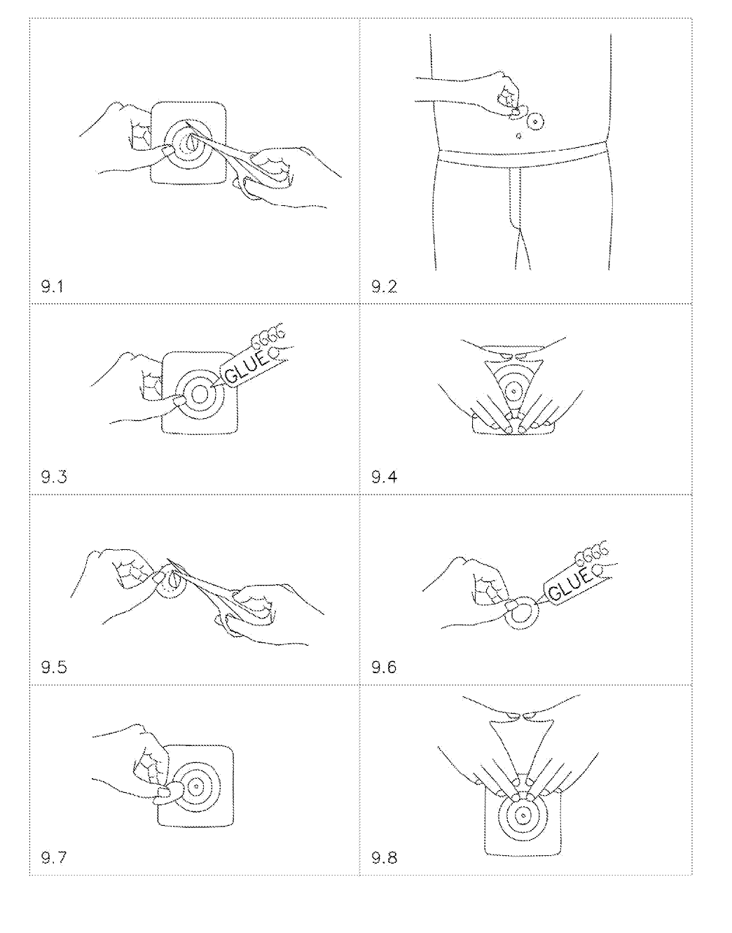

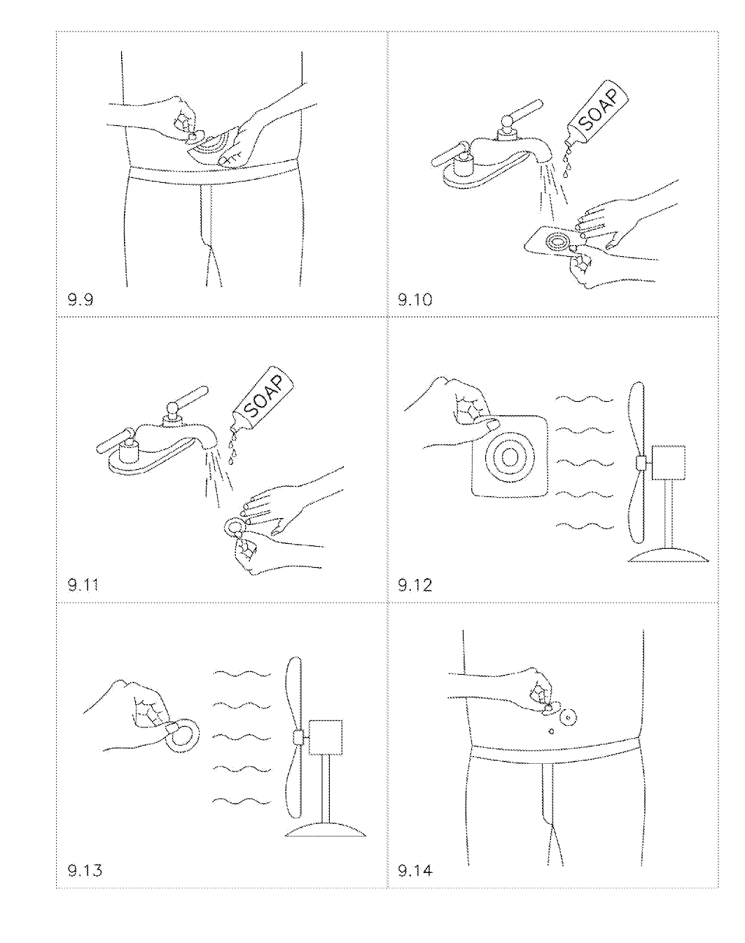

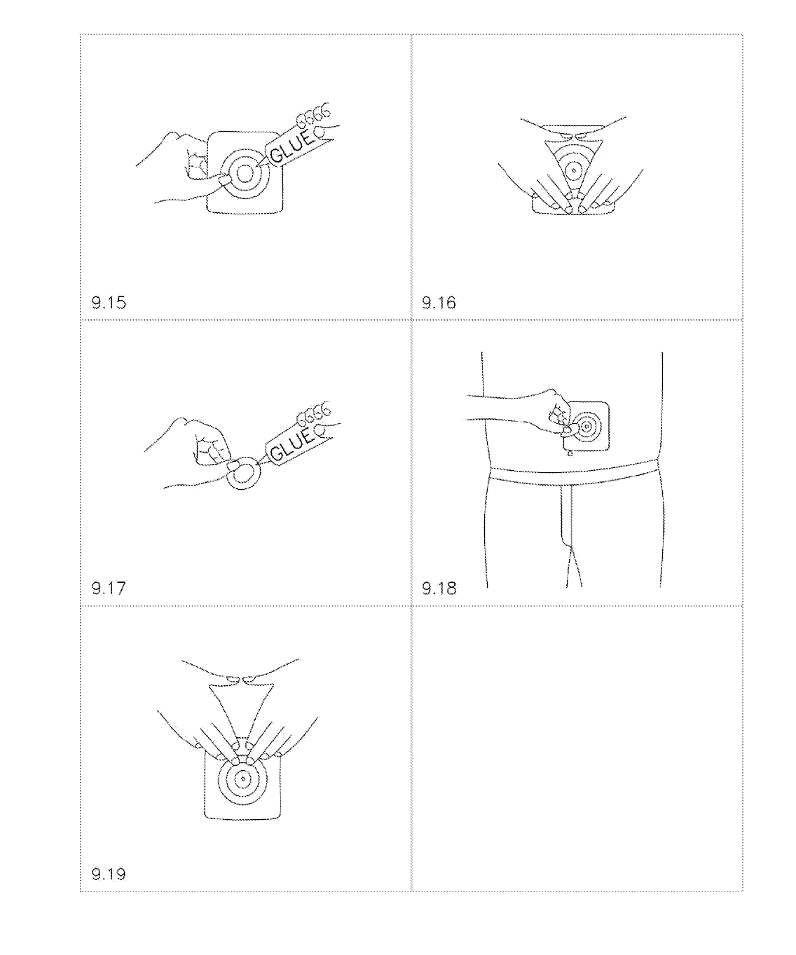

9.1) Cutting out the center of the washable wafer to the size of the stoma.

9.2) Applying the adhesive prep as liquid or as a spray to prepare the skin around the stoma.

9.3) Applying the washable liquid or spray adhesive to the back of the washable stoma wafer to form an adhesive backing to apply on a stoma.

9.4) Applying the washable stoma wafer with the applied adhesive or glue backing on a stoma.

9.5) Cutting out the center of the elastic stoma barrier ring to the size of the stoma.

9.6) Applying the adhesive spray prep as liquid or spray adhesive to the back of the elastic stoma barrier with the center cut out.

9.7) Applying the adhesive prep as liquid or as a spray to prepare the skin around the stoma center cut out of the washable stoma wafer now adhering on the skin and applying at the same time the same adhesive prep to purposely overlap onto the plastic surface around the wafer center cut out by a surface area at least the width of the prepared elastic stoma barrier ring in step 6 above.

9.8) Applying the elastic stoma barrier ring with the applied adhesive to adhere around the exposed stoma skin and to the washable stoma wafer center cut out surface purposely overlapped with adhesive prep by a surface area at least the width of the elastic stoma barrier ring.

9.9) Removing the washable stoma wafer with the elastic stoma barrier ring from the skin around the stoma after use, using physical means and with the stoma adhesive remover.

9.10) Washing the washable stoma wafer.

9.11) Washing the elastic stoma barrier ring.

9.12) Drying the washed, washable stoma wafer.

9.13) Drying the washed elastic stoma barrier ring.

9.14) Applying the adhesive prep as liquid or spray to prepare the skin around the stoma.

9.15) Applying new washable liquid or spray adhesive to form a fresh new adhesive backing on the washed and dried washable stoma wafer.

9.16) Applying new washable liquid or spray adhesive to form a fresh new adhesive backing on the washed and dried washable stoma wafer.

9.17) Applying the washed and dried washable stoma wafer with the new adhesive backing on a stoma.

9.18) Applying the adhesive prep as liquid or spray around the center cut out of the washable wafer adhering on the skin around the stoma and to purposely overlap onto the plastic or elastic stoma wafer center by a surface area at least the width of the elastic stoma barrier ring.

9.19) Applying the washed and dried elastic stoma barrier ring with the new adhesive backing to adhere around the stoma skin exposed and to the washable stoma wafer center cut-out surface purposely overlapped with adhesive prep by a surface area at least the width of the elastic stoma barrier ring.

### DRAWINGS DETAILED EMBODIMENT

1) Colostomy pouch rear view of two-piece pouch and stoma wafer separate.
2) Colostomy pouch front view of two-piece pouch and stoma wafer separate.
3) Impermeable water resistant material.
4) Snap-on ring.
5) Belt strap handles.
6) Holding or handling handle.
7) “Velcro” style closing mechanism of pouch.
8) Clamp style closing mechanism of pouch.
9) Entry point of body waste.
10) Colostomy pouch rear view of one-piece pouch and washable stoma wafer together.
11) Colostomy pouch front view of one-piece pouch and washable stoma wafer together.
12) Rear view of washable stoma wafer of a one-piece pouch and washable stoma wafer together.
13) Impermeable plastic or elastic center of stoma wafer cut-out section.
14) Stoma wafer cut-out to fit around a stoma.
15) Adhesive absorbing surface material.
16) Rear view of washable stoma wafer of a two-piece pouch and stoma wafer separate
17) Front view of washable stoma wafer of a two-piece stoma pouch and washable stoma wafer separate.
18) Stoma opening cut-out.

### DETAILED DESCRIPTION OF THE INVENTION

The present invention relates to the method by which colostomy, ileostomy, urostomy, and other similar stoma wafers also known as stoma flanges or stoma skin barriers equipped with attached bag as a one piece unit collecting the body wastes in the bag or colostomy and other similar stoma wafers equipped with detached bag collecting the body wastes in the bag having the body waste collecting bag attach to the stoma wafer by a snap-on or other methods of attachment for colostomy or other similar applications involving stomas that the stoma wafers are manufactured with a center stoma cut-out that is made of plastic material or material similar to plastic surrounded by a band of washable flexible material or fabric onto which adhesives are generally applied from one (1) to six (6) inches in band width or that the stoma wafers manufactured with a center stoma cut-out that is made of elastic material or other material with similar elasticity surrounded by a band of washable flexible material or fabric onto which adhesives are generally applied from one (1) to six (6) inches in band width are produced or manufactured for the purpose of this invention method without an adhesive backing, that the elastic or plastic center of the stoma wafer is cut out to the diameter size of the stoma, that the skin around the stoma is prepared by applying liquid or spray adhesive prep, that the adhesive or glue in the form of liquid or spray that could be adhesive glue or adhesive prep in the form of liquid or spray or other adhesive type that is washable with hand soap, alcohol, stoma adhesive remover, or other cleaning agent is applied to the washable stoma wafer at the time of use on a stoma thus creating a fresh adhesive backing for the washable stoma wafer, that the washable stoma wafer with plastic or elastic center cut-out with fresh adhesive backing is applied and pressed to stick to the skin around the stoma that has been prepared by coating with adhesive prep,

that the skin exposed around the stoma cut-out of the stoma wafers with a plastic or similar material to plastic center stoma cut-out is coated with the same adhesive prep in the form of liquid or spray as the adhesives used on the skin and to overlap with the same adhesive prep onto the plastic surface around the stoma wafer center cut-out by a surface area at least the width of the elastic stoma barrier ring

that the center cut-out of an elastic or other material with similar elasticity stoma barrier ring is cut out to the diameter size of the stoma that the cut elastic stoma barrier ring is coated on the back with the same type of adhesive as the adhesives used for the backing of the washable stoma wafer to form a fresh new washable adhesive backing, that the elastic stoma barrier ring with the fresh new washable adhesive backing is applied and pressed to stick firmly to the exposed skin around the stoma and to the plastic around the stoma that has been coated with adhesive prep to assure the seal,

that the stoma wafers with elastic or other material with similar elasticity center stoma cut-out not require a stoma barrier ring because they have a snug impermeable fit around a stoma not exposing the skin around the stoma,

that the washable elastic stoma wafer without a stoma barrier ring or the washable plastic stoma wafer with an elastic stoma barrier ring that has been applied and pressed and used for the maximum period of time possible or maximum time required will be removed from the skin around the stoma by physical means and with stoma adhesive remover and also by using the same stoma adhesive remover or to remove the used adhesive residue on the skin from the washable stoma wafer removed, that the elastic or plastic center-cut-out wafer and the elastic stoma skin barrier ring will be washed using the appropriate cleaner that could be hand soap, alcohol, stoma adhesive remover, or other cleaning agent, will be dried, will have fresh washable adhesive applied to the same washable elastic or plastic center cut-out washed stoma wafer and to the same washable washed elastic stoma skin barrier ring to form a fresh new washable adhesive backing to apply again as a washable elastic or plastic center cut-out washable stoma wafer and an elastic stoma skin barrier ring with a fresh washable adhesive backing to apply on a stoma and to repeat this method or process until the washable elastic or plastic center cut-out stoma wafer and the washable elastic stoma barrier ring become unusable.

### Claims

The invention relates to the method by which colostomy, ileostomy, urostomy, and other similar stoma wafers also called stoma flanges or stoma skin barriers with a center stoma cut-out made of plastic or similar material, requiring a stoma barrier ring or a stoma wafer with a center cut-out made of elastic or similar material, not requiring a stoma barrier ring, either of which plastic or elastic stoma wafer center cut-out surrounded by a band of washable flexible material or fabric onto which adhesives are generally applied from one (1) to six (6) inches in band width having an attached body waste collecting pouch as a one-piece undetached wafer and pouch unit collecting the body wastes in the pouch or colostomy and other similar stoma wafers with a plastic or elastic center stoma cut-out having a detached two-piece snap-on or other methods of attachment wafer and pouch unit collecting the body wastes in the pouch for colostomy or other similar applications involving stomas in that:

What is claimed is:

1. That the stoma wafer, that has a center stoma cut-out made of plastic or similar material or of elastic or similar material surrounded by flexible material or fabric onto which adhesive is generally applied, washable by hand soap, alcohol, stoma adhesive remover or other cleaning agent, be produced as a final product of use without glue or adhesive as adhesive backing or with a thin layer of glue washable with hand soap, alcohol, stoma adhesive remover or other cleaning agent that will not increase the thickness of the stoma wafer without the glue.

2. That the stoma wafer, that has a center stoma cut-out made of plastic or similar material or of elastic or similar material surrounded by flexible material or fabric onto which adhesive is generally applied, washable by hand soap, alcohol, stoma adhesive remover or other cleaning agent that has been produced as a final product of use without glue or adhesive as adhesive backing, or with a thin layer of glue washable with hand soap, alcohol, stoma adhesive remover or other cleaning agent that will not increase the thickness of the stoma wafer without the glue be cut at the inner plastic or elastic center to the diameter size of the stoma onto which it is applied.

3. That the stoma wafer, that has a center stoma cut-out made of plastic or similar material or of elastic or similar material surrounded by material or fabric onto which adhesive is generally applied, washable by hand soap, alcohol, stoma adhesive remover or other cleaning agent that has been produced as a final product of use without glue or adhesive as adhesive backing that has been cut at the inner plastic or elastic center to the diameter size of the stoma to be applied on, be coated with a glue or an adhesive in the form of liquid or spray that is washable with hand soap, alcohol, stoma adhesive remover or other cleaning agent to form an adhesive backing.

4. That the adhesive prep in the form of liquid or spray that is washable with hand soap, alcohol, stoma adhesive remover or other cleaning agent be applied to the skin.

5. That the stoma wafer that has a center stoma cut-out made of plastic or similar material or of elastic or similar material surrounded by material or fabric onto which adhesive is generally applied, washable by hand soap, alcohol, stoma adhesive remover or other cleaning agent that has been produced as a final product of use without glue or adhesive as adhesive backing or with a thin layer of glue washable with hand soap, alcohol, stoma adhesive remover or other cleaning agent, that has been cut at the inner plastic or elastic center to the diameter size of the stoma, that has been coated with a glue or an adhesive in the form of liquid or spray that is washable with hand soap, alcohol, stoma adhesive remover or other cleaning agent to form an adhesive backing or the stoma wafer that is initially made with a thin layer, be applied on the skin around the stoma that has been coated with adhesive prep.

6. That If the washable stoma wafer, without glue or adhesive backing or with a thin layer of glue washable with hand soap, alcohol, stoma adhesive remover or other cleaning agent that will not increase the thickness of the stoma wafer without the glue .that has a center stoma cut-out made of plastic or similar material surrounded by flexible material or fabric onto which adhesives are generally applied, that are washable by hand soap, alcohol, stoma adhesive remover, or other cleaning agent used, that the washable plastic stoma wafer that has been produced as a final product of use without glue or adhesive as adhesive backing or with a thin layer of glue washable with hand soap, alcohol, stoma adhesive remover or other cleaning agent, that has been cut out at the inner plastic center to the diameter size of the stoma onto which it is applied that has been coated with a glue or an adhesive in the form of liquid or spray that is washable by hand soap, alcohol, stoma adhesive remover or other cleaning agent that has been applied on the skin around a stoma, that has had the skin around the stoma pre-coated with adhesive prep that a flat thin, less than 1/32″ of an inch in thickness elastic or similar material stoma barrier ring be cut out in the center to the size of the stoma circumference be cut with a wide enough outer circumference to cover the exposed skin around the center plastic wafer cut-out and to overlap onto the plastic surface of the washable stoma wafer, that the prepared cut elastic stoma barrier ring be coated on one side with an adhesive washable by hand soap, alcohol, stoma adhesive remover, or other cleaning agent to form an adhesive backing, that the skin exposed at the center cut out of the washable wafer plastic around the stoma be coated with adhesive prep and to purposely overlap with the same adhesive prep onto the plastic surface around the stoma wafer center cut-out by a surface area at least the width of the elastic stoma barrier ring, that the prepared cut stoma barrier ring coated on one side with adhesive be applied to the skin around the stoma and to the washable stoma wafer plastic surface coated with adhesive prep to create an impermeable barrier between the stoma and the washable stoma wafer plastic surface.

7. That if the stoma wafer without glue or adhesive backing or with a thin layer of glue washable with hand soap, alcohol, stoma adhesive remover or other cleaning agent that will not increase the thickness of the stoma wafer without the glue has a center stoma cut-out made of elastic or other material with similar elasticity that is cut to the diameter size of the stoma onto which it is applied, that is coated with a glue or an adhesive in the form of liquid or spray that is washable with hand soap, alcohol, stoma adhesive remover or other cleaning agent to form an adhesive backing applied on the skin around the stoma, the elastic stoma wafer will not require the use of a stoma barrier ring.

8. That the stoma wafer, with a plastic or elastic center cut-out produced as a final product of use without glue or adhesive as adhesive backing with a thin layer of glue washable with hand soap, alcohol, stoma adhesive remover or other cleaning agent that will not increase the thickness of the stoma wafer without the glue or the elastic stoma barrier ring that has been coated with a glue or an adhesive in the form of liquid or spray that is washable with hand soap, alcohol, stoma adhesive remover or other cleaning agent to form an adhesive backing that has been applied on a stoma, will be washed with soap while still adhering on the skin by showering, bathing, or whatever other similar hygiene method, will be dried by using a hair dryer or other similar means while still adhering to the skin, will have fresh adhesive applied on the stoma wafer surfaces that have lifted or unglued and to the skin surfaces directly in front of the wafer membrane surfaces that have lifted, and to repeat the process of washing the wafer with soap while on the skin, drying the stoma wafer while on the skin and applying the same adhesive again to the stoma wafer surfaces that lift off the skin and to the skin surfaces directly in front of the stoma wafer surfaces that have lifted off the skin until the stoma wafer is removed completely form the skin using physical means and stoma adhesive remover to be washed with hand soap, alcohol, stoma adhesive remover or other cleaning agent.

9. That the stoma wafer, with a plastic or elastic center cut-out produced as a final product of use without glue or adhesive as adhesive backing or with a thin layer of glue washable with hand soap, alcohol, stoma adhesive remover or other cleaning agent that will not increase the thickness of the stoma wafer without the glue, or the elastic stoma barrier ring that has been coated with a glue or an adhesive in the form of liquid or spray that is washable with hand soap, alcohol, stoma adhesive remover or other cleaning agent to form an adhesive backing that has been applied on a stoma, will be removed from the skin around the stoma using physical means and stoma adhesive remover and will be washed with hand soap, alcohol, stoma adhesive remover or other cleaning agent.

10. That the stoma wafer, with a plastic or elastic center cut-out produced as a final product of use without glue or adhesive as adhesive backing or with a thin layer of glue washable with hand soap, alcohol, stoma adhesive remover or other cleaning agent that will not increase the thickness of the stoma wafer without the glue or the elastic stoma barrier ring that has been coated with a glue or an adhesive in the form of liquid or spray that is washable with hand soap, alcohol, stoma adhesive remover or other cleaning agent to form an adhesive backing that has been applied on a stoma that has been removed from the skin around the stoma using physical methods and stoma adhesive remover after use that has been washed with hand soap, alcohol, stoma adhesive remover, or other cleaning agent, will be dried by methods similar to drying clothing and fabric.

11. That the stoma wafer, with a plastic or elastic center cut-out produced as a final product of use without glue or adhesive as adhesive backing or with a thin layer of glue washable with hand soap, alcohol, stoma adhesive remover or other cleaning agent that will not increase the thickness of the stoma wafer without the glue that has been coated with a glue or an adhesive in the form of liquid or spray that is washable with hand soap, alcohol, stoma adhesive remover, or other cleaning agent if not present initially, to form an adhesive backing that has been be applied on a stoma that has been removed after use from the skin around the stoma using physical methods and stoma adhesive remover, that has been washed with hand soap, alcohol, stoma adhesive remover, or other cleaning agent and that has been dried by regular methods similar to drying clothing and fabric, that the same washable stoma wafer has fresh glue or adhesive applied to it in the form of liquid or spray that is washable with hand soap, alcohol, stoma adhesive remover or other cleaning agent, to form a new adhesive backing.

12. That the stoma wafer, with a plastic or elastic center cut-out produced as a final product of use without glue or adhesive as adhesive backing or with a thin layer of glue washable with hand soap, alcohol, stoma adhesive remover or other cleaning agent that will not increase the thickness of the stoma wafer without the glue, that has been coated with a glue or an adhesive in the form of liquid or spray that is washable with hand soap, alcohol, stoma adhesive remover, or other cleaning agent to form an adhesive backing if nor present initially, that has been applied on a stoma, that has been removed from the skin around the stoma using physical means and stoma adhesive remover that has been washed with hand soap, alcohol, stoma adhesive remover, or other cleaning agent that has been dried by regular methods of drying clothing and fabric and that has had fresh glue or adhesive applied to it in the form of liquid or spray that is washable with hand soap, alcohol, stoma adhesive remover or other cleaning agent to form a new adhesive backing, will be applied on a stoma, and the method or procedure described herein be a method or procedure to be performed repeatedly until the washable plastic or elastic stoma wafer or the elastic stoma barrier ring eventually become unusable.
